(19) United States
(12) Patent Application Publication
(10) Pub. No.: US 2025/0082941 A1
(43) Pub. Date: Mar. 13, 2025

(54) MEASURING CARDIAC ELECTRICAL PROPAGATION USING SINGLE LEAD CONFIGURATION

(71) Applicant: Impulse Dynamics NV, Willemstad (CW)

(72) Inventors: David PRUTCHI, Voorhees, NJ (US); Tamir BEN DAVID, Tel-Aviv (IL)

(73) Assignee: Impulse Dynamics NV, Willemstad (CW)

(21) Appl. No.: 18/828,025

(22) Filed: Sep. 9, 2024

Related U.S. Application Data
(60) Provisional application No. 63/536,945, filed on Sep. 7, 2023.

(51) Int. Cl. A61N 1/365; A61N 1/05; A61N 1/39

(52) U.S. Cl. CPC A61N 1/365; A61N 1/3628; A61N 1/39622; A61N 1/056

(57) ABSTRACT

An implantable cardiac device comprises a device can and a single lead extending from the device can for cardiac emplacement. The lead senses a first cardiac wave propagating along a first vector and the same cardiac wave propagating along a second vector. The first vector may be between first and second locations on the lead, and the second vector between the lead and the device can or involving other locations on the lead so that a second lead is not needed and it is possible to make a single lead device.

### RELATED APPLICATION(S)

This application claims the benefit of priority under 35 USC § 119(e) of U.S. Provisional Patent Application No. 63/536,945 filed on Sep. 7, 2023, the contents of which are incorporated herein by reference in their entirety.

### FIELD OF THE INVENTION

The present invention, in some embodiments thereof, relates to an implantable cardiac device (ICD), and, more particularly, but not exclusively, to an implantable Cardiac Contractility Modulation device, which may or may include a defibrillation coil. The implantable cardiac devices (ICD) may include wearable ICDs and subcutaneous ICDs.

### BACKGROUND OF THE INVENTION

Implanted cardiac devices, such as pacemakers and defibrillators, use electrical leads to detect signals from heart muscle activity at different locations. However, the leads used by such an implantable cardiac device may result in interference with venous blood flow and tricuspid valve function. Accordingly, it is helpful to minimize the number of leads.

The current technique for measuring the cardiac tissue electrical velocity and direction in implantable devices is to use two separate leads that are placed at two different locations in the heart, typically in the right ventricle (RV).

One therapy that uses such an implantable device is Cardiac Contractility Modulation. Cardiac Contractility Modulation is generally understood as a therapy which is intended for the treatment of patients with moderate to severe heart failure (New York Heart Association (NYHA) class II-IV) with symptoms despite optimal medical therapy, who can benefit from an improvement in cardiac output. The short- and long-term use of this therapy enhances the strength of ventricular contraction and therefore the heart's pumping capacity by modulating (adjusting) the myocardial contractility. This is provided by a pacemaker-like device that applies non-excitatory electrical signals adjusted to and synchronized with the electrical action in the cardiac cycle.

In Cardiac Contractility Modulation therapy, electrical stimulation is applied to the cardiac muscle during the absolute refractory period. In this phase of the cardiac cycle, electrical signals cannot trigger new cardiac muscle contractions, hence this type of stimulation is known as a non-excitatory stimulation. However, the electrical signals increase the influx of calcium ions into the cardiac muscle cells (cardiomyocytes). In contrast to other electrical stimulation treatments for heart failure, such as pacemaker therapy or implantable cardioverter defibrillators (ICD), Cardiac Contractility Modulation does not directly affect cardiac rhythm. Rather, the aim is to enhance the heart's natural contraction, the native cardiac contractility, sustainably over long periods of time. Furthermore, unlike most interventions that increase cardiac contractility, Cardiac Contractility Modulation is not associated with an unfavorable increase in oxygen demand by the heart, which increase may be measured in terms of

Myocardial Oxygen Consumption or MVO₂. This may be explained by the beneficial effect the therapy has in improving cardiac efficiency. A meta-analysis in 2014 and an overview of device-based treatment options in heart failure in 2013 concluded that Cardiac Contractility Modulation treatment is safe, that it is generally beneficial to patients and that the treatment increases the exercise tolerance (ET) and quality of life (QoL) of patients. Furthermore, preliminary long-term survival data shows that Cardiac Contractility Modulation is associated with lower long-term mortality in heart failure patients when compared with expected rates among similar patients not treated with Cardiac Contractility Modulation.

In some implantable devices, such as the Cardiac Contractility Modulation device, the time difference between the peak of the R wave as detected by the two leads is used for the decision if to give Cardiac Contractility Modulation stimulation or not.

### SUMMARY OF THE INVENTION

The present embodiments may provide a way to select appropriate cardiac beats for delivering Cardiac Contractility Modulation stimulation using a single lead configuration of the implanted device. That is to say, a single lead configuration may correctly identify proper cardiac beats, during which stimulation may safely be applied.

In some configurations of Cardiac Contractility Modulation therapy, there is a need to measure two separate ECG vectors in different orientations to identify an eligible heart beat for stimulation, and the present embodiments may provide a solution therefor that uses a single lead.

According to an aspect of some embodiments of the present invention there is provided an implantable cardiac device comprising a device can and a single lead extending from the device can for cardiac emplacement, the lead configured to sense a first a first cardiac wave propagating along a first vector and a same cardiac wave propagating along a second vector, said first and second vectors having respectively different directions.

In embodiments, at least said first vector is sensed between first and second locations on said lead.

Embodiments may calculate for each signal a time of the R wave peak and obtain a difference (ΔT) therebetween.

Embodiments may compare said difference (ΔT) to a predetermined interval, or to a predetermined interval range, to identify qualifying heart beats during which Cardiac Contractility Modulation stimulation may be safely applied.

Embodiments may apply Cardiac Contractility Modulation stimulation when said difference lies within said predetermined interval or said predetermined interval range and not to apply Cardiac Contractility Modulation stimulation when said difference lies outside said predetermined interval or said predetermined interval range.

Embodiments may update said predetermined interval or said predetermined interval range following a change, consistent for a predetermined number of instances, of said difference (ΔT), or consistent for a predetermined time interval.

In embodiments, said lead comprises a tip at a distal end thereof and a ring, the ring being placed behind said tip relative to said distal end, and said first and second locations comprise said tip and said ring.

Embodiments may use a vector between said ring and said device can for said first or second vector.

Embodiments may include a first implantable cardioverter defibrillator (ICD) shock coil or a defibrillation electrode located on said lead.

Embodiments may select a vector for said first or second vector that lies between said first ICD shock coil or a defibrillation electrode and said device can.

Embodiments may include a second ICD shock coil located on said lead, the device being configured to select a vector for said first or second vector that lies between said second ICD shock coil and said device can or the lead tip or ring.

In embodiments, said lead comprises a tip, a ring, a first ICD shock coil and optionally a second ICD shock coil and wherein said first and second vectors may be defined by any two of:
said tip to said ring;
said tip to said first ICD shock coil;
said tip to said second ICD shock coil;
said tip to said device can;
said ring to said first ICD shock coil;
said ring to said second ICD shock coil;
said ring to said device can;
said first ICD shock coil to said second ICD shock coil;
said first ICD shock coil to said device can; and
said second ICD shock coil to said device can.

The single lead may have a lead tip at a distal end, and the lead tip may be located in a right ventricle of a recipient.

According to a second aspect of the systems and methods described herein there may be provided a method of sensing two non-parallel ECG vectors using an implanted cardiac device having a single lead placed within the heart, the method comprising:
sensing a first cardiac wave propagating along a first vector using said single lead; and
measuring said first cardiac wave propagating along a second vector using said single lead, said first vector and said second vector being respectively different vectors.

The first vector may be between first and second locations on said lead.

The method may involve comparing said first and second cardiac propagations to identify a qualifying heartbeat for providing Cardiac Contractility Modulation.

The method may involve identifying a qualifying heartbeat comprises calculating for each signal a time of the R wave peak and obtaining a difference (ΔT) therebetween.

The method may involve calculating a correlation between said first and said second signals.

In embodiments, identifying a qualifying heartbeat may include comparing said difference (ΔT) to a predetermined interval or interval range.

The method may involve applying said Cardiac Contractility Modulation stimulation when said difference lies within said predetermined interval or interval range and not applying Cardiac Contractility Modulation stimulation when said difference lies outside said predetermined interval or interval range.

The method may involve updating said predetermined interval or interval range following a change, consistent for a predetermined number of instances, of said difference (ΔT).

In the method, said lead may have a tip and a ring, the ring being placed distally from said tip, and one of the vectors may be between the tip and the ring.

The method may involve providing a first implantable cardioverter defibrillator (ICD) shock coil on said lead.

The method may involve selecting an axis for said first or second vector that lies between said first ICD shock coil and said device can or the lead tip or ring.

The method may involve providing a second ICD shock coil on said lead.

The method may involve selecting an axis for said first or second vector that lies between said second ICD shock coil and said device can or the lead tip or ring.

In the method, said selecting said first and second vectors may involve finding a combination of members of said group providing a best signal for measuring said time difference (ΔT).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified diagram showing the R wave measurements obtained using the present embodiments against a reference of a surface PQRS complex;

FIG. 2 is a simplified diagram showing how the measured R wave timings may relate to a preset condition for carrying out Cardiac Contractility Modulation according to the present embodiments;

FIG. 3 is a simplified diagram illustrating the implanted device according to the present embodiments;

FIG. 4 is a detail of the tip of the electrode of FIG. 3;

FIG. 5 is a version of the diagram of FIG. 3 showing the bipolar electrode to can electrocardiogram vector;

FIG. 6 is a version of the diagram of FIG. 3 showing a lead with a coil according to embodiments of the present invention;

FIG. 7 is a variation of the device of FIG. 6 showing a lead with two coils according to embodiments of the present invention; and

FIG. 8 is a flow chart showing operation of the implantable device during successive heartbeats.

### DESCRIPTION OF SPECIFIC EMBODIMENTS OF THE INVENTION

The present invention, in some embodiments thereof, relates to an implantable cardiac device, and an implantable lead for detecting heart signals, which is connected to the body of the device, hereinafter the can, and, more particularly, but not exclusively, to an implantable Cardiac Contractility Modulation device, which may or may not include an ICD coil for defibrillation.

An implantable cardiac device according to the present embodiments may include a device can and a single lead extending from the device can for cardiac emplacement. The lead measures a first cardiac signal along a first vector and a second cardiac signal along a second vector. The first vector may be between first and second locations on the lead, and the second vector between the lead and the device can or involving other locations on the lead so that a second lead is not needed and it is possible to make a single lead device.

Thus, the lead is a single lead. Due to the configuration of the lead it is possible to obtain a suitable time difference that is equivalent to the difference in times of measurement of the peak of the R wave by the two leads, thus identifying the absolute refactory period.

As an alternative to the time difference, an embodiment may measure a correlation change between the two signals, for example by calculation of a cross-correlation coefficient.

Thus, the lead of the present embodiments is an implantable single lead having an intracardiac bipolar electrode.

The device uses the lead to measure two different electrocardiogram signals. A first electrocardiogram signal is a signal measured at the bipolar electrode. A second electrocardiogram signal is a signal measured between one of the electrode poles and the device housing. The device then uses the two measurements to obtain the timing of the peak of the R wave in each case, T1 and T2 and finds the difference ΔT. The Cardiac Contractility Modulation stimulation is provided, during the absolute refactory period, provided that ΔT is within a predetermined time interval or range, which interval or range may vary according to the patient and according to normal measurements of the heart rate, obtained say when the device is implanted.

In embodiments, the single lead may include one or more ICD coils. The second signal may thus be obtained in one of two ways. The first way is from between the ICD coil and one of the electrode poles. The second possibility is to obtain the signal from between the ICD coil and the device can or housing.

Accordingly, an implantable cardiac device comprises a device can and a single lead extending from the device can for cardiac emplacement. The lead measures a first cardiac signal along a first vector and a second cardiac signal along a second vector. The first vector is between first and second locations on the lead, so that a second lead is not needed and it is possible to make a single lead device.

More particularly, in the present embodiments, two vectors are used along with a single lead configuration, to measure the cardiac electrical potential difference between two points that are located on the lead. As will be explained in greater detail below the two points may be any of the tip, the ring, the first defibrillation coil, the second defibrillation coil (if present) and the device can or housing.

The combinations that may be used for individual vectors are:
Tip to Ring
Tip to 1st coil
Tip to 2nd coil
Tip to device can
Ring to 1st coil
Ring to 2nd coil
Ring to device can
1st coil to 2nd coil
1st Coil to device can
2nd Coil to device can.

As will be discussed in greater detail below, the combination of vectors to use may depend on the individual implantation and an initialization procedure may be used to identify the vectors to be used.

In some cases, a defibrillation electrode rather than a coil may be present, and all references to a defibrillation coil herein are to be understood as extending to a defibrillation electrode.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

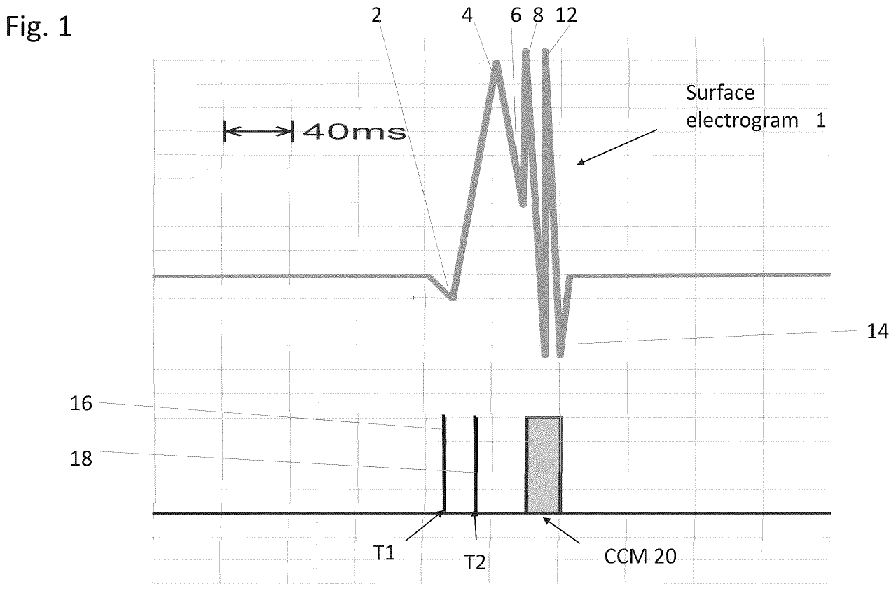

Referring now to the drawings, FIG. 1 illustrates the timing of the R wave detection using two different vectors, with the surface electrogram (ECG) 1. The surface electrogram is typically taken from the skin surface, and is shown as a reference in the upper part of the diagram. Each rectangle represents 40 milliseconds. At the beginning of the signal corresponding to ventricular activation and Cardiac Contractility Modulation stimulation delivery, the reference signal shows an initial fall 2 (“Q wave”), the first trough, and then a rise to a first peak 4 (“R wave”). Following the first peak there is a part fall 6 in the signal, followed sometimes by additional peaks (e.g. 8 and 12) that result from possible artifact of the Cardiac Contractility Modulation stimulation.

The timing of the local R wave peak in the first signal 16 is shown as T1 and may approximately correspond to the first trough 2 (commonly in the Q-R segment) in the surface signal 1. The timing 18 of the R wave peak in the second signal is shown as T2. T2 may fall closer or farther away from T1 depending on electrode positioning, cardiac tissue substrate condition, medication, etc. Provided that the time difference between T1 and T2 fulfils a preset condition, then Cardiac Contractility Modulation 20 is provided during the absolute refractory period. The absolute refractory period is a period when the heart muscle does not respond to stimulation with a new activation, and is thus a time when Cardiac Contractility Modulation can be provided safely without causing any reactivation to the ventricles. The absolute refactory period is thus the CCM period 20, and is followed by the relative refactory period.

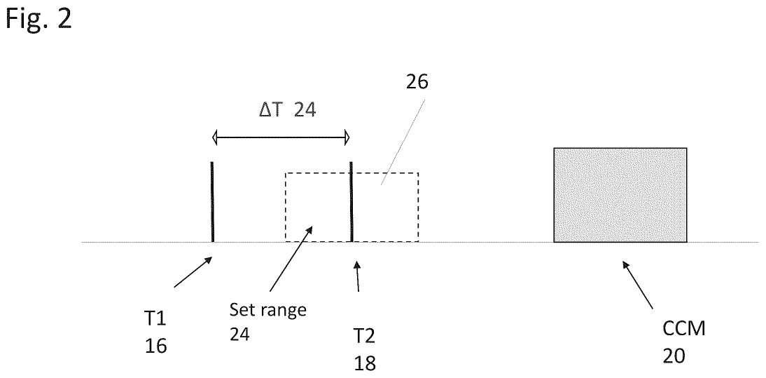

Reference is now made to FIG. 2, which shows in greater detail the set range of ΔT 24 where T2 18 must fall for carrying out Cardiac Contractility Modulation 20 during the absolute refractory period. As explained, the times T1 16 and T2 18 are obtained by measuring the times of the R wave in two different vectors. ΔT 24 is simply the time difference between the two times. Box 26, shown in hashed lines, is a set range, which is commonly programmable. If ΔT lies within the set range 24, then Cardiac Contractility Modulation 20 is carried out. If ΔT lies outside the range, it may be a sign that an abnormal beat is occurring, and such condition may be a sign of arrythmia in which case Cardiac Contractility Modulation should not be applied.

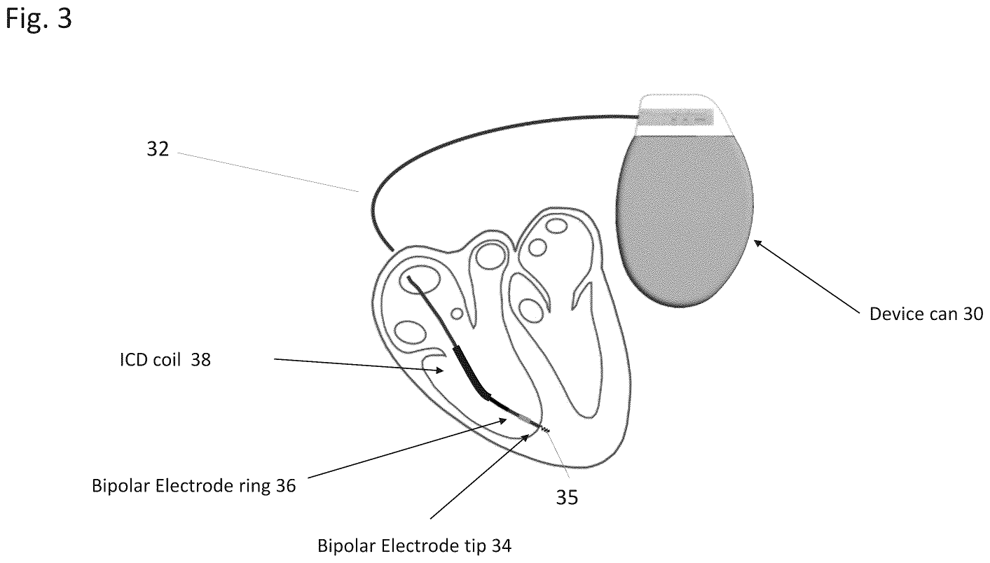

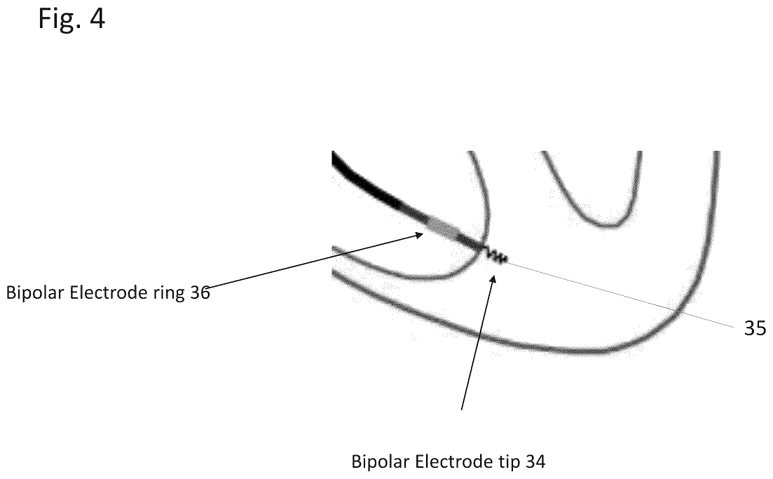

Referring now to FIG. 3, the device that is implanted includes a housing or can 30 and a lead 32 which extends from the housing into the region of the heart. The lead 32 includes a bipolar electrode tip 34, which may be helically-shaped—35, a bipolar electrode ring 36, and an ICD shock coil 38. The ICD shock coil 38, as mentioned above, provides for the delivery of high-voltage shocks for defibrillation when the heartbeat is abnormal, as opposed to Cardiac Contractility Modulation which is applied when the heartbeat is normal. The end of the electrode 32 with the helical bipolar tip 34 and the bipolar electrode ring are shown enlarged in FIG. 4.

The term “bipolar electrode” is used herein to refer to an electrode pair which, although formed by single electrodes, has two sensing configurations to make two different and distinguishable measurements from two different vectors.

In FIG. 3 the lead has one ICD coil 38, which delivers shocks to help treat life-threatening arrhythmias that can cause sudden cardiac arrest. Other embodiments below have additional coils. In this configuration, time T1 may be detected from the signal measured between the tip 34 of the lead and electrode ring 36. Time T2 however is measured using another signal measured at a different vector, for example between the electrode tip 34 or the electrode ring 36 or the device coil 38 and the device can 30, or any other suitable vector that may be available, including making use of the device coil 38. In some embodiments the selection of a vector for T2 may be made after the placement of the lead in the heart.

Absolute values of ΔT may vary depending on the vector used, but calibration may be used to find a normal range. Furthermore, depending on the position of the lead, different choices of vector for T2 may be appropriate. Calibration may be carried out initially, after placement of the device in the body. T1 is measured as well as T2 and an initial ΔT is calculated. The calibration may include testing of different vectors for T2 followed by selection of the vector giving the results suited to the application.

It is further noted that longer term changes in ΔT may result from electrode movement inside the body, so longer term modifications of ΔT may be incorporated into a self-modifying algorithm. For example, a predetermined number of consistently different ΔT measurements may trigger the selection of a modified ΔT range. Thus, the initial calibration may be continually modified.

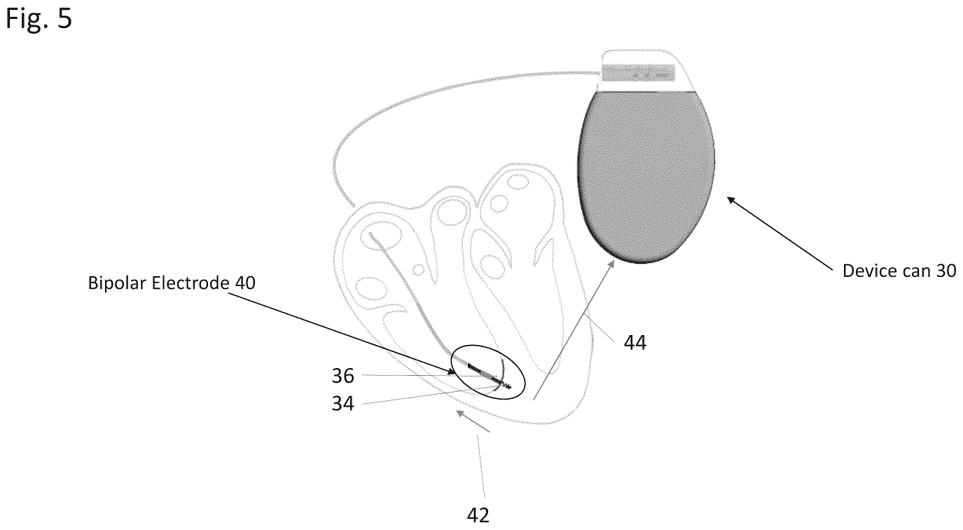

Reference is now made to FIG. 5, which is a version of FIG. 3 showing the bipolar electrode 40 having lead tip 34 and ring 36. In the embodiment of FIG. 5, there is no ICD shock coil. T1 is detected from the change in potential difference across the vector between the tip and the ring—the vector being illustrated by arrow 42, and T2 is measured between the bipolar electrode 40 and the can 30, the vector being illustrated by arrow 44.

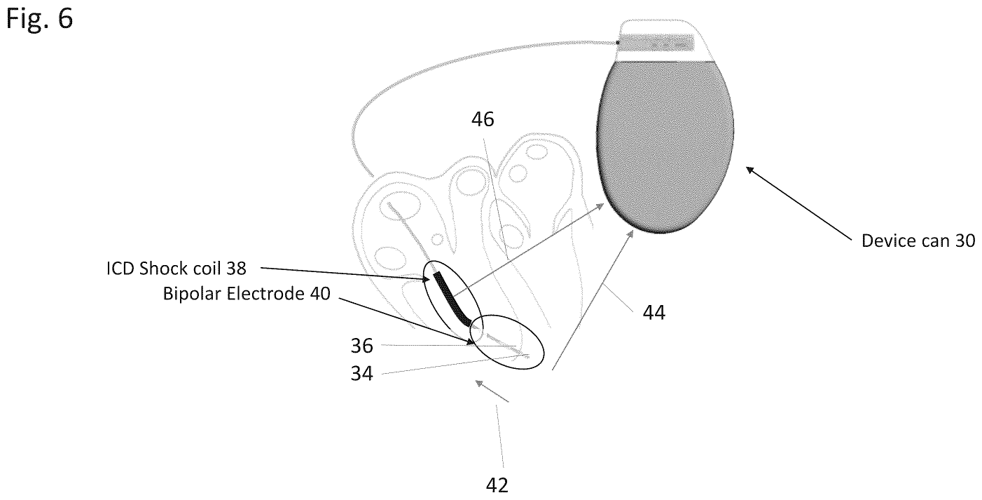

Reference is now made to FIG. 6, which is a further embodiment of the implantable device of FIG. 3, including an ICD shock coil 38. The bipolar electrode 40 has helical tip 34 and ring 36. Tl is detected from the change in potential difference across the vector between the tip and the ring—the vector being illustrated by arrow 42, and T2 may be measured as before across the vector between the bipolar electrode tip and the can, the vector being illustrated by arrow 44. However, unlike with FIG. 5, there is now an additional available vector for T2, namely the coil 38 to can 30 electrocardiogram vector being illustrated by arrow 46.

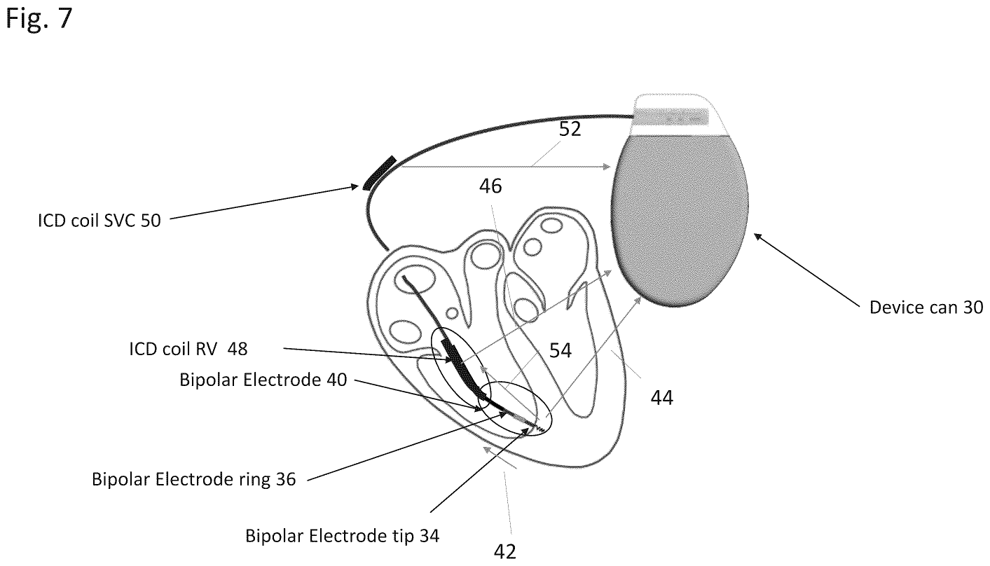

Reference is now made to FIG. 7, which is a simplified diagram of a further embodiment of an implantable device according to the present invention. Bipolar electrode 40 includes a tip 34 and ring 36 and then there are two ICD shock coils, a right ventricle coil 48 and a coil 50 placed in the superior vena cava (SVC). The additional ICD shock coil provides an additional vector for measuring T2, namely the potential difference across the vector between itself and the can 30, shown as vector 52.

Likewise T2 may be measured between one of the coils and a node or pole on the electrode. For example vector 54 between the RV ICD coil 48 and the bipolar electrode ring 36 may be used.

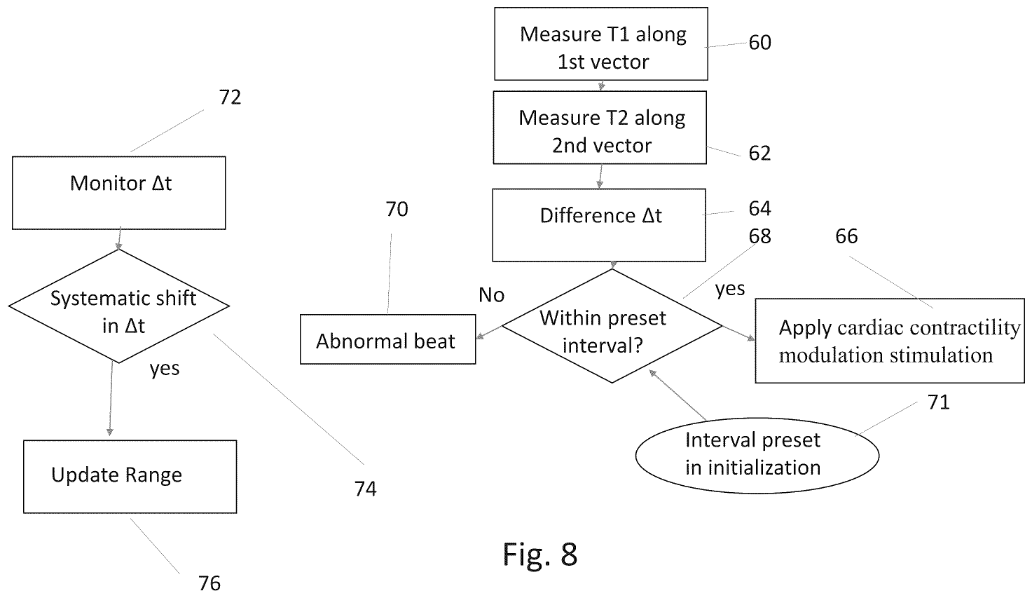

The present embodiments may thus provide a Cardiac Contractility Modulation device with an implantable single lead having an intracardiac bipolar electrode. Referring now to FIG. 8, the device may measure a first electrocardiogram signal, from the first vector—box 60 and a second electrogram signal—box 62. As discussed above, there are several vectors that may be selected for the first and second measurements.

The device measures the timing of the R wave (T1, T2) with respect to the first and second signals and calculates the timing difference ΔT—box 64. The device provides Cardiac Contractility Modulation stimulation—box 66 only in the case where ΔT is within a set range—decision box 68. If the difference is outside the range then an abnormal beat is assumed—box 70—and no action is taken.

As per the cases of FIGS. 6 and 7 above, in the case where the single lead also includes one or more ICD coils, the second signal can be measured between:
The ICD coil and one of the electrode poles, say vector 54.
The ICD coil and the device can—vectors 46 and 52.

In some embodiments, the set range is set during an initialization procedure—box 71. Following implantation, there may be continual monitoring of ΔT during use to measure any changes that persist during normal cardiac activity—box 72, and when a systematic ΔT shift is detected—box 74, the set range may be updated—box 76. In some cases, conditions like elevation of heart rate or increase of patient activity may cause systematic ΔT shift. Any update of set range may accordingly take into consideration such conditions. To avoid effect of such conditions on ΔT shift detection, the shift evaluation—box 74, may be carried out during rest and at a heart rate below a set value, typically below 80 heart beats per minutes. Alternatively, the set value may be the daily average heart rate or the daily average heart rate less the daily average heart rate standard deviation.

As explained, there are multiple vectors that may be selected using the single lead and the device can. The combination of vectors employed may depend on the specific placement of the device and the lead during implantation. The initialization procedure may identify the best combination of two vectors to give the best signal for determining the time difference ΔT. The initialization may involve running through all of the vectors and/or combinations of vectors to find which gives the best signal in the specific case.

It is expected that during the life of a patent maturing from this application many relevant implantable electrodes and devices will be developed and the scopes of these and other terms herein are intended to include all such new technologies a priori.

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An implantable cardiac device comprising a device can and a single lead extending from said device can for cardiac emplacement, the lead configured to detect propagation of a cardiac wave along a first vector and propagation of said cardiac wave along a second vector, said first and second vectors having respectively different directions.

2. The implantable cardiac device of claim 1, wherein at least said first vector extends between first and second locations on said lead.

3. The implantable cardiac device of claim 1, further configured to calculate for each signal a time of the R wave peak and obtain a difference (ΔT) therebetween.

4. The implantable cardiac device of claim 3, configured to compare said difference (ΔT) to a predetermined interval, or to a predetermined interval range, to identify qualifying heart beats during which Cardiac Contractility Modulation stimulation may be safely applied.

5. The implantable cardiac device of claim 4, configured to apply Cardiac Contractility Modulation stimulation when said difference lies within said predetermined interval or said predetermined interval range and not to apply Cardiac Contractility Modulation stimulation when said difference lies outside said predetermined interval or said predetermined interval range.

6. The implantable cardiac device of claim 3, configured to update said predetermined interval or said predetermined interval range following a change, consistent for a predetermined number of instances, of said difference (ΔT), or consistent for a predetermined time interval.

7. The implantable cardiac device of claim 3, wherein said lead comprises a tip at a distal end thereof and a ring, the ring being placed behind said tip relative to said distal end, and wherein said first and second locations comprise said tip and said ring.

8. The implantable cardiac device of claim 7, configured to use a vector between said ring and said device can for said first or second vector.

9. The implantable cardiac device of claim 7, further comprising a first implantable cardioverter defibrillator (ICD) shock coil or a defibrillation electrode located on said lead.

10. The implantable cardiac device of claim 9, configured to select a vector for said first or second vector that lies between said first ICD shock coil or a defibrillation electrode and said device can.

11. The implantable cardiac device of claim 9, comprising a second ICD shock coil located on said lead, the device being configured to select a vector for said first or second vector that lies between said second ICD shock coil and said device can or the lead tip or ring.

12. The implantable cardiac device of claim 1, wherein said lead comprises a tip, a ring, a first ICD shock coil and optionally a second ICD shock coil and wherein said first and second vectors are selected from any two members of the group consisting of:
said tip to said ring;
said tip to said first ICD shock coil;
said tip to said second ICD shock coil;
said tip to said device can;
said ring to said first ICD shock coil;
said ring to said second ICD shock coil;
said ring to said device can;
said first ICD shock coil to said second ICD shock coil;
said first ICD shock coil to said device can; and
said second ICD shock coil to said device can.

13. The implantable cardiac device of claim 1, said single lead having a lead tip at a distal end, said lead tip being located in a right ventricle of a recipient.

14. A method of measuring two non-parallel ECG vectors using an implanted cardiac device having a single lead placed within the heart, the method comprising:
sensing propagation of a first cardiac wave along a first vector using said single lead; and
sensing propagation of said first cardiac wave along a second vector using said single lead, said first vector and said second vector being respectively different vectors.

15. The method of claim 14, wherein at least the first vector is between first and second locations on said lead.

16. The method of claim 14, comprising comparing said first and second cardiac signals to identify a qualifying heartbeat for providing Cardiac Contractility Modulation.

17. The method of claim 16, wherein said identifying a qualifying heartbeat comprises calculating for each signal a time of the R wave peak and obtaining a difference (ΔT) therebetween.

18. The method of claim 14, comprising calculating a correlation between said propagations along said first and second vectors respectively.

19. The method of claim 16, wherein said identifying a qualifying heartbeat further comprises comparing said difference (ΔT) to a predetermined interval or interval range.

20. The method of claim 19, comprising applying said Cardiac Contractility Modulation stimulation when said difference lies within said predetermined interval or interval range and not applying Cardiac Contractility Modulation stimulation when said difference lies outside said predetermined interval or interval range.

21. The method of claim 19, comprising updating said predetermined interval or interval range following a change, consistent for a predetermined number of instances, of said difference (ΔT).

22. The method of claim 14, wherein said lead comprises a tip and a ring, the ring being placed distally from said tip, and wherein one of the vectors is between the tip and the ring.

23. The method of claim 14, further comprising providing a first implantable cardioverter defibrillator (ICD) shock coil on said lead.

24. The method of claim 23, comprising selecting an axis for said first or second vector that lies between said first ICD shock coil and said device can or the lead tip or ring.

25. The method of claim 23, comprising providing a second ICD shock coil on said lead.

26. The method of claim 25, comprising selecting an axis for said first or second vector that lies between said second ICD shock coil and said device can or the lead tip or ring.

27. The method of claim 14, wherein said lead comprises a tip, a ring a first ICD shock coil and optionally a second ICD shock coil and wherein said first and second vectors are selected from any two members of the group consisting of:
said tip to said ring;
said tip to said first ICD shock coil;
said tip to said second ICD shock coil;
said tip to said device can;
said ring to said first ICD shock coil;
said ring to said second ICD shock coil;
said ring to said device can;
said first ICD shock coil to said second ICD shock coil;
said first ICD shock coil to said device can; and
said second ICD shock coil to said device can.

28. The method of claim 27, wherein said selecting said first and second vectors comprises finding a combination of members of said group providing a best signal for measuring said time difference (ΔT).

29. The method of claim 14, said single lead having a lead tip at a distal end, the method comprising placing said lead tip in a right ventricle of a recipient.
